(19) United States
(12) Patent Application Publication
(10) Pub. No.: US 2026/0325233 A1
(43) Pub. Date: Oct. 22, 2026

(54) MEDICAL NAVIGATION METHOD, MEDICAL NAVIGATION SYSTEM, AND COMPUTER PROGRAM

(71) Applicant: NATIONAL INSTITUTE OF ADVANCED INDUSTRIAL SCIENCE AND TECHNOLOGY, Tokyo (JP)

(72) Inventors: Ryousuke TSUMURA, Ibaraki (JP); Kiyoshi YOSHINAKA, Ibaraki (JP); Yoshihiko KOSEKI, Ibaraki (JP)

(73) Assignee: NATIONAL INSTITUTE OF ADVANCED INDUSTRIAL SCIENCE AND TECHNOLOGY, Tokyo (JP)

(21) Appl. No.: 19/120,391

(22) PCT Filed: Oct. 11, 2023
(86) PCT No.: PCT/JP2023/036831

§ 371 Date: Apr. 11, 2025

(30) Foreign Application Priority Data
Oct. 14, 2022 (JP) 2022-165133

(51) Int. Cl. A61B 34/30; A61B 7/02; A61B 8/00; A61B 34/20

(52) U.S. Cl. CPC A61B 34/30; A61B 7/02; A61B 8/4263; A61B 2034/2055; A61B 2034/2065

(57) ABSTRACT

A medical navigation method for guiding a medical device to an appropriate position on a body surface, in accordance with individual difference between body shapes, is provided. the medical navigation method comprises: a step for holding three-dimensional point cloud information of a subject as body model data of the subject (S102), holding N pieces of comparison model data and arrangement data for the medical device in each of the pieces of the comparison model data, and determining reference model data that has the highest degree of similarity between it and the body model data of the subject (S106); a step for setting, as a target, the body model data of the subject, and applying registration using a non-rigid ICP algorithm to the reference model data (S108); and a step for converting, by using a coordinate transformation formula, obtained as a result of the registration, for the three-dimensional point cloud information between the body model data of the subject and the reference model data, the arrangement data in the reference model data to arrangement data for the medical device in the body model data of the subject (S110).

### TECHNICAL FIELD

The present invention relates to a medical navigation method, a medical navigation system, and a computer program.

### BACKGROUND ART

In the case that a clinical specialist such as a physician or the like remotely manipulates a medical device such as an ultrasound probe, a stethoscope, or the like via a medical robot or the like, it is necessary to guide the medical robot or the like to make it know a position, on a body surface, on which a medical device should be positioned, for efficiently performing inspection. Further, in the case that a physician offers telemedicine, there may be a case wherein a person such as a patient himself/herself or a close relative of the patient who does not have medical knowledge, instead of the physician, is required to arrange a medical device such as a stethoscope or the like on an appropriate position on a body surface of the patient. On the other hand, a position where a medical device should be arranged, that is, a position of an organ in a patient who is a subject of inspection, is different from those of others according to individual differences between body shapes of patients, and, accordingly, an important matter is position estimation that takes individual differences into consideration.

For example, each of Patent Literatures 1 and 2 and Non-patent Literature 1 discloses a method wherein a standard model of a brain of a human is applied to an image of a brain of a patient for alignment and deformation (registration) thereof, for mapping of functional parts of a brain.

### CITATION LIST

### Patent Literature

PTL 1: U.S. Pat. No. 8,311,359

PTL 2: Japanese Patent Application Public Disclosure No. 2019-535472

### Non Patent Literature

NPL 1: Brian B Avants, Nicholas J Tustison, Michael Stauffer, Gang Song, Baohua Wu, and James C Gee, “The Insight ToolKit image registration framework,” Front Neuroinform, Frontiers Research Foundation, Switzerland, Apr. 28, 2014

### SUMMARY OF INVENTION

### Technical Problem

However, in the above-explained prior-art technique, brain images, which are uniform in terms of shapes when compared with body shapes of patients, are used; and, further, medical image modality that requires imaging means such as a CT (Computer Tomography), an MRI (Magnetic Resonance Imaging), or the like is required for performing registration.

In view of the above matters, an object of the present invention is to provide a medical navigation method, a medical navigation system, and a computer program which make it possible to guide a medical device such as an ultrasound probe, a stethoscope, or the like to an appropriate position on a body surface, according to individual differences of body shapes, by using an RGB-D (image/depth) camera which can be used conveniently.

### Solution to Problem

For achieving the above object, a mode of the present invention comprises a medical navigation method performed by a computer system for arranging a medical device on a subject, wherein the computer system holds three-dimensional point cloud information of the subject as body model data of the subject, and holds N pieces of comparison model data showing human body shapes, and arrangement data showing a position(s) for arrangement of the medical device in each of the pieces of the comparison model data, and the medical navigation method comprises: a step for determining a piece of the comparison model data that has the highest degree of similarity between it and the body model data of the subject, in the N pieces of the comparison model data, as reference model data; a step for setting, as a target, the body model data of the subject, and applying registration using a non-rigid ICP (interactive closest point) algorithm to the reference model data; and a step for converting, by using a coordinate transformation formula, obtained as a result of the registration, for the three-dimensional point cloud information between the body model data of the subject and the reference model data, the arrangement data in the reference model data to arrangement data for the medical device in the body model data of the subject.

Further, the other mode of the present invention comprises a medical navigation system for arranging a medical device on a subject; for holding three-dimensional point cloud information of the subject as body model data of the subject, and holding N pieces of comparison model data showing human body shapes, and arrangement data showing a position(s) for arrangement of the medical device in each of the pieces of the comparison model data; determining a piece of the comparison model data that has the highest degree of similarity between it and the body model data of the subject, in the N pieces of the comparison model data, as reference model data; setting, as a target, the body model data of the subject, and applying registration using a non-rigid ICP (interactive closest point) algorithm to the reference model data; and converting, by using a coordinate transformation formula, obtained as a result of the registration, for the three-dimensional point cloud information between the body model data of the subject and the reference model data, the arrangement data in the reference model data to arrangement data for the medical device in the body model data of the subject.

Further, a different mode of the present invention comprises a computer program which makes a computer system perform the above-explained medical navigation method.

Further, a different mode of the present invention comprises a computer-readable storage medium in which a computer program which makes a computer system perform the above-explained medical navigation method is stored.

### BRIEF DESCRIPTION OF DRAWINGS

FIG. 1 is a figure which shows an example of a construction of a medical robot device (a medical navigation system) according to an embodiment of the present invention.

FIG. 2 is a figure which shows an outline of a medical navigation method according to an embodiment of the present invention.

FIG. 3 is a figure which shows human body models constructed by performing simulation.

FIG. 4 is a figure which shows all results of registration errors corresponding to chamfer distances.

FIG. 5 is a figure which shows an example of a process flow of a medical navigation method according to an embodiment of the present invention.

### DESCRIPTION OF EMBODIMENTS

In the following description, embodiments of the present invention will be explained in detail with reference to the figures. A medical navigation method and a medical navigation system according to the present embodiment are a method and a system for appropriately estimating, when applying medical activity to a subject such as a patient or the like by using a medical device such as an ultrasound probe, a stethoscope, or the like in a medical robot device, an arrangement position of the medical device on a body surface of the subject. In this regard, in the following description, as an example, a case wherein the medical device is a stethoscope and auscultation with respect to a specific part in a body of a patient (a subject) is performed will be explained.

### (Construction of Medical Robot Device)

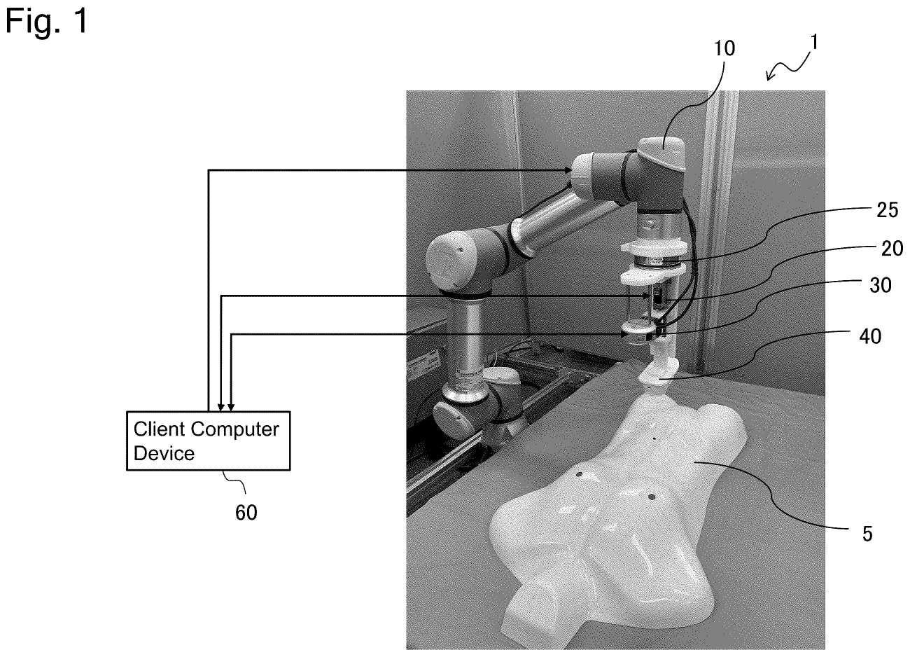

FIG. 1 is a figure which shows an example of a construction of a medical robot device (a medical navigation system) which performs a medical navigation method according to a preset embodiment. A medical robot device 1 according to the present embodiment comprises a robot arm 10, a constant force passive scan mechanism (an end effector) 20, and an RGB-D camera 30. The robot arm 10 is constructed to be able to move around a patient (a subject) who is an object of medical activity (FIG. 1 is that used for explanation, and, herein, a mannequin is substituted for a patient 5 (Specifically, the patient 5 means a body of the patient 5. The same hereinafter, and it may also be referred to as the “patient body 5”)). Further, the constant force passive scan mechanism (the end effector) 20 is attached to a tip part of the robot arm 10 in an integral or attachable/detachable manner. The constant force passive scan mechanism (the end effector) 20 is a mechanism which holds the medical device 40, and makes it possible to put and press the medical device 40 on the body surface of the patient 5 and move it while maintaining application of a certain load. In this regard, the medical device refers to any of devices in general which are necessary when performing medical activity applied to the patient 5, such as an ultrasound probe, a stethoscope, and so on, for example. More specifically, an example of a medical device which can be used in the medical robot device 1 according to the present embodiment is an electronic stethoscope such as an electronic stethoscope (a digital stethoscope) JPES-01 from Mitorika Co., Ltd. Sound collected by putting the electronic stethoscope into contact with the patient 5 may be transmitted as data to a computer device (such as a client computer device 60 which will be explained later). Similarly, other medical devices may also be constructed to be able to transmit data of sound, images, and so on, that are obtained by putting the medical devices into contact with the patient 5, to the computer device. Further, the robot arm 10 is able to have, on its tip part, the RGB-D camera 30 in an integral or attachable/detachable manner. The RGB-D camera 30 is a camera which is able to obtain, at the same time, a color image (RGB information) and a range image (range information).

Further, the medical robot device 1 may comprise a client computer device 60. The client computer device 60 is directly or indirectly connected to each of the robot arm 10, the constant force passive scan mechanism (the end effector) 20, and the RGB-D camera 30 in the medical robot device 1, and performs data transmission/reception operation with each of the above components. For example, the client computer device 60 transmits control signals for controlling the respective components, and transmits data required for activating the respective components. Further, the client computer device 60 receives, from the respective components, data obtained or generated by the respective components. The client computer device 60 may be realized by using a hardware construction similar to that of a general computer device. For example, the client computer device 60 comprises a processor, a RAM (Random Access Memory), a ROM (Read Only Memory), a built-in hard disk device, a removable memories such as an external hard disk device, a CD, a DVD, a USB memory, a memory stick, an SD card, and so on, input/output devices (a display, a keyboard, a mouse, a touch panel, a speaker, a microphone, an LED, and so on), a wired/wireless communication interface which allows communication with the respective components in the medical robot device 1 and other computer devices, and so on. For example, in the client computer device 60, a processor is able to read, into a memory such as a RAM or the like, a computer program which has been stored in advance in a hard disk device, a ROM, a removable memory, or the like and makes the client computer device 60 perform a medical navigation method according to the present embodiment; and performs operation and reads required data from the hard disk device, the ROM, the removable memory, or the like when appropriate during the operation. As a result of operation, such as that explained above, of the client computer device 60, various kinds of processes in the medical robot device 1 explained in the present embodiment can be realized. Further, various kinds of pieces of data used in respective processes explained in relation to the present embodiment are stored in a storage device or a storage medium such as a hard disk device, a RAM, a removable memory or the like, and read and used by the processor as necessary during the time when processing is performed.

Further, the medical robot device 1 shown in FIG. 1 is a medical robot device which was actually constructed by the inventors listed in this application. As the robot arm 10, a 6-axis collaborative robot arm (UR5e, Universal Robot, Denmark) was used. As the RGB-D camera 30, a LIDAR camera (L515 RealSense, Intel, USA) which obtains a three-dimensional contour of the body surface of the patient 5 as point cloud data was used. Further, as the client computer device 60, a publicly known computer device (Dell Precision 5380, Dell, USA) was used. Further, the medical robot device 1 in the present example was constructed on the assumption that auscultation is performed by using a stethoscope, for example. In this regard, since the medical robot device shown in FIG. 1 is that constructed experimentally, a mannequin is substituted for the patient 5 herein, and, since nipples and a navel which are used as landmarks on the patient 5 in the present example, that will be explained later, are indistinct in the case of a mannequin, markers are added to positions of the nipples and the navel by the inventors. Further, the construction shown in FIG. 1 is a mere example, and, of course, the construction of the medical robot device 1 according to the present embodiment is not limited to that in the above example. Matters similar to the above matters also apply to the whole of the specification.

Further, in the construction example in FIG. 1, in view of safety of medical inspection of the patient 5, the end effector 20 in the robot arm 10 is provided with a constant force passive scan mechanism which uses a spring for adaptively grasping the stethoscope (the medical device) 40 to apply it to the body surface of the patient 5. It is constructed in such a manner that a 6-axis force and torque sensor 25 (Axia-80-M20, ATI Industrial Automation, USA) is attached to a base of the constant force passive scan mechanism (the end effector) 20, and contact force at the time when the stethoscope 40 is put on the body surface of the patient 5 is measured. Further, the client computer device 60 for controlling the medical robot device 1 and an external server computer device (which is not shown in the figure) are directly connected to a network (the speed is 1 GB/sec), and the data transmission protocol thereof is TCP/IP. In this regard, although the RGB-D camera 30 is installed in the constant force passive scan mechanism (the end effector) 20 in this example, the construction is not limited to that shown in the above example. The RGB-D camera 30 may be fixed in such a manner that its position relative to the robot arm 10 does not change, and arranged in a position different from that of the constant force passive scan mechanism (the end effector) 20.

The RGB-D camera 30 obtains, as point cloud data, information of the depth and the color of the shape of the patient 5, and the point cloud data is used for registration (alignment) of coordinates that uses positional relationship between the robot arm 10 and the patient 5. Since the RGB-D camera 30 is attached to the end effector 20 in the robot arm 10, positional relationship between the RGB-D camera 30 and the robot arm 10 is kinematically fixed even if the robot arm 10 is moved around the patient 5. Accordingly, positions of the obtained point cloud data of the patient 5 are linked to coordinates of the robot arm 10.

Further, the robot arm 10 may be controlled by using a programming language, URScript (Universal Robots A/S), used in the medical robot device 1. The client computer device 60 for controlling the medical robot device 1 may transmit, to an external server computer device (which is not shown in the figure), commands in URScript via socket communication. Further, the point cloud data is obtained by using Intel RealSence SDK 2.0. A software system, which is customized based on Python programming of Visual Studio Code, is able to achieve synchronization between controlling of the robot arm 10 and reading of the point cloud data.

### (Outline of Medical Navigation Method)

In the medical navigation method according to the present embodiment, for localizing an area(s) of auscultation performed by using a stethoscope in the state that individual difference between body shapes of patients is compensated, body surface registration between the patient body 5 and a reference model of a body, that has been registered with the medical navigation system, is used. In this regard, the “reference model” is a model of a human body which is regarded as the reference for body surface registration. In the reference model, an auscultation position(s) (this includes a case wherein the position is an area having a certain size (an auscultation area), and the same applies hereinafter) on which the stethoscope should be arranged has been determined in advance based on general medical knowledge of a clinical specialist or the like, and arrangement data representing the auscultation position(s) has been stored in the medical navigation system. In the body surface registration in the present embodiment, correspondence relationship between the patient body 5 and the reference-model surface is calculated. Based on the correspondence relationship calculated by using the body surface registration, the auscultation area(s) that has been identified in the reference model may be projected to the body of the patient.

In this regard, one of registration methods that have been generally known is an ICP (interactive closest point) algorithm. This makes it possible to apply, for fitting a reference model to the body surface of a patient, input point cloud data of a body surface by using affine transformation; and, as a result, the point cloud is regarded as a rigid body (rigid). On the other hand, since the body surfaces differ according to respective individuals, it is not possible to find accurate correspondence relationship between surfaces of the patient body 5 and the reference-model in the case of the rigid registration according to the IPC, and it is necessary to deform the input point cloud data to fit in with the body surface of the target (the patient body 5).

Accordingly, in the medical navigation method and the medical navigation system according to the present embodiment, a non-rigid ICP, which is able to make the point cloud data fit in with the patient body 5 in a non-rigid manner by using feature points and deformation constraints, is used as an improved ICP algorithm. In the present embodiment, by applying body surface registration between the patient 5 and the reference model, an auscultation position(s) in the patient body 5 is identified. More specifically, it becomes possible to improve accuracy of localization of the auscultation area(s), by preparing, in advance, multiple human-body models (comparison models) which may become several types of reference models, and selecting a comparison model which closely resembles the body of the patient. In each comparison model, an auscultation position(s) (this includes a case wherein the position is an area having a certain size (an auscultation area), and the same applies hereinafter) on which the stethoscope should be arranged has been determined in advance based on general medical knowledge of a clinical specialist or the like, and arrangement data representing the auscultation position(s) has been stored in the medical navigation system.

Further, in the present embodiment, a case wherein positions of four valves (the aortic valve, the pulmonary valve, the tricuspid valve, and the mitral valve) that are necessary to know for auscultation of a heart of a patient (a subject) will be explained as an example. The aortic valve, the pulmonary valve, the tricuspid valve, and the mitral valve are generally positioned on a lower left side of the sternum close to the fifth intercostal space, the left fifth intercostal apex on the midclavicular line (approximately 10 cm from the midline), the inner edge of the left second intercostal space, and the right third intercostal space, respectively. However, it is difficult for a person who is not a medical professional to correctly identify them. The medical navigation method and the medical navigation system according to the present embodiment estimate, from a reference model, the position corresponding to each valve on the body surface of the patient (the position on which a stethoscope should be arranged), by applying non-rigid registration between the patient body 5 and the reference model of the human body that has been registered in advance with the medical navigation system.

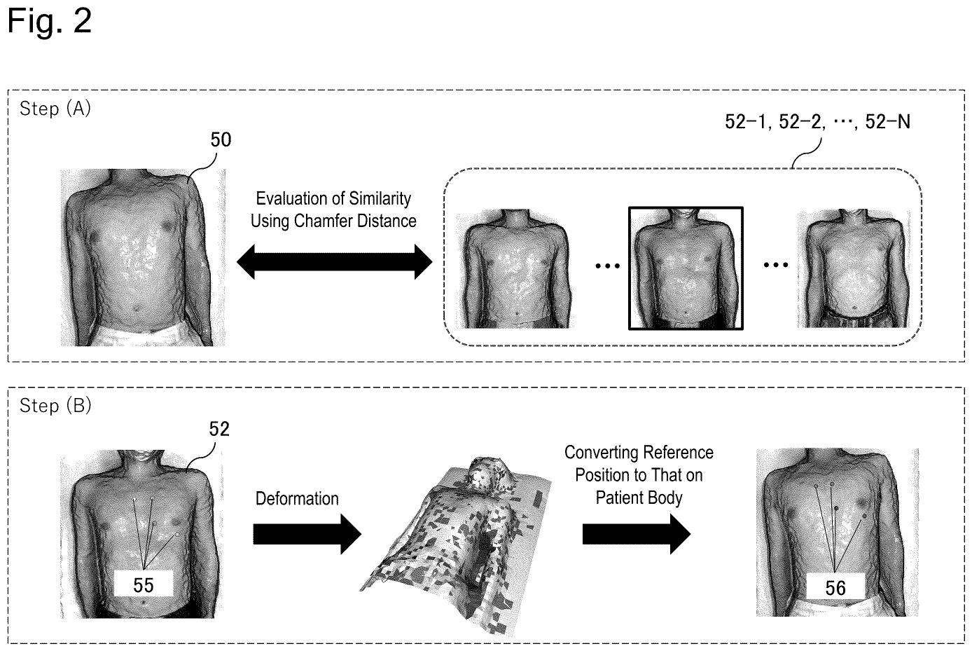

FIG. 2 is a figure which shows an outline of the medical navigation method according to the present embodiment. As shown in FIG. 2, the medical navigation method according to the present embodiment comprises two processing steps, step (A) and step (B). In step (A), from multiple comparison models 52-1, 52-2, . . . , and 52-N (N is an integer equal to or greater than 2) which have been stored in advance in the medical navigation system and are bases of reference models, a comparison model which most closely resembles patient body data (a patient body model) obtained by taking an image of the patient body 5 by the RGB-D camera 30 is select as a reference model 52. In step (B), in the reference model 52 selected in step (A), body surface registration is performed by using non-rigid ICP to deform the reference model 52 to fit in with the patient body 5 (the patient body model 50). As a result, the position (a reference position) 55 of each valve is projected from the reference model 52 to the patient body 5 (the patient body model 50), and, accordingly, the position (an estimated position) 56 of each valve on the body surface of the patient body 5 is estimated.

(Step (A): Selection of Comparison Model which Most Closely Resembles Body of Patient)

In the following description, an example of the process in step (A) will be explained in detail. For selecting a comparison model which most closely resembles the patient body 5, the point cloud data of the patient body model 50 and the point cloud data of each of the comparison models 52-k (k: an integer in 1-N) are superimposed with each other, and the degree of superimposition is calculated, in order to measure similarity between the patient body model 50 and each of the comparison models 52-k. Chamfer distance is widely adopted for measuring similarity between two point sets, and is defined as follows:

$$\begin{matrix}( FORMULA 1 ) & \\ {d}_{CD} ( {S}_{1} , {S}_{2} ) = {\sum }_{x ∈ {S}_{2}} {min}_{y ∈ {S}_{2}} ‖ x - y ‖ + {\sum }_{y ∈ {S}_{2}} {min}_{x ∈ {S}_{2}} ‖ x - y ‖ & Formula ( 1 )\end{matrix}$$

$${S}_{1} , {S}_{2} ∈ {ℝ}^{3}$$

In this regard, S₁ and S₂ are subsets of point cloud data. x and y are point data included in S₁ and S₂. d_{CD} represents a chamfer distance between S₁ and S₂. d_{CD} may be calculated by summing up the squared distances between nearest neighbor correspondences of two point clouds. In the case that the deviation in terms of shapes between the two point clouds is large, the distances between the nearest neighbor correspondences of the two point clouds become large, and, accordingly, the chamfer distances become large. By calculating the chamfer distance between the patient body model 50 and each of the comparison models 52-k, a comparison model 52-k (the reference model 52) which most closely resembles the patient body model 50 is identified.

For calculating the chamfer distance, subsets, which have different shapes and arrangements, of point clouds are roughly superimposed first. Next, by using ICP registration, alignment (registration) of two subsets is performed, roughly based on local features of the point cloud. In each of the subsets, a fast point feature histograms (FPFH) feature quantity may be calculated; and correspondence relationship between the feature quantities of the two subsets may be searched for by using RANSAC (Random Sample Consensus).

With respect to all comparison models 52-k, the chamfer distances are calculated as the degrees of similarity between them to which the ICP registration has been applied and the patient body model 50. The most closely resembling comparison model 52-k (the reference model 52) will be used in the non-rigid ICP process that will be explained later.

### (Step (B): Non-Rigid ICP for Body Surface Registration)

Non-rigid ICP is used for deforming the reference model 52, that has been identified in step (A), to fit in with the patient body model 50. In the present embodiment, the non-rigid ICP was applied to mesh data converted from the dot cloud data. The source mesh converted from the point cloud data of the reference model 52 is given as a set V of n vertices (n is a natural number equal to or greater than 3) and a set e of m edges (m is a natural number equal to or greater than 3). In the registration, detecting of detection parameters X, that represent a set V (X) of displaced source vertices that constitute a deformed mesh corresponding to the body surface of the patient 5, is performed. For obtaining the parameters X, an objective function E was defined as follows:

(FORMULA 2)

E(X):=E_{d}(X)+αEₛ(X)+βEₗ(X) Formula (2)

In this regard, E_{d}, Eₛ, and Eₗ represent a distance objective function, a stiffness objective function, and a landmark distance objective function, respectively. α and β represent stiffness and a landmark parameter. If corresponding source vertices and target vertices that are vertices in a target mesh converted from point cloud data of the patient body model 50 is defined as (vᵢ, uᵢ), the distance objective function is defined as follows:

$$\begin{matrix}( FORMULA 3 ) & \\ {E}_{d} ( X ) := {\sum }_{{v}_{i} ∈ 𝒱} {w}_{i} {‖ {X}_{i} {v}_{i} - {u}_{i} ‖}^{2} & Formula ( 3 )\end{matrix}$$

Here, the degree of reliability with respect to matching is weighted by Wᵢ. If no corresponding vertex exists, the weight is set to zero. If a corresponding vertex is found, the weight is set to “1.” The stiffness objective function is used to normalize deformation, by imposing a penalty on weighted difference of transformations of neighbor vertices. The stiffness objective function is defined by using a Frobenius norm

∥·∥_{F} (FORMULA 4)
and a weighting matrix G, as shown below:

$$\begin{matrix}( FORMULA 5 ) & \\ {E}_{s} ( X ) := {\sum }_{\{ i , j \} ∈ ℰ} {‖ ( {X}_{i} - {X}_{j} ) G ‖}_{F}^{2} & Formula ( 5 )\end{matrix}$$

In this regard, G was used for weighting a difference between a rotational part and a skewed part of deformation to the translational part of the deformation. Finally, the landmark distance objective function was used for initialization and guidance of the registration, and the landmark distance objective function was defined as follows:

$$\begin{matrix}( FORMULA 6 ) & \\ {E}_{i} ( X ) := {\sum }_{( {v}_{i} , l ) ∈ ℒ} {‖ {X}_{i} {v}_{i} - l ‖}^{2} & Formula ( 6 )\end{matrix}$$

In this regard,

[symbol]

={(vᵢ,l₁), . . . (vᵢ_{ₗ},lₗ)} (FORMULA 7)
represents a set of landmarks for mapping source vertices to the target vertices. In the present embodiment, nipples and a navel are used as landmarks for matching the reference model 52 with a body surface of the upper half of the body of the patient 5. Thereafter, before applying the non-rigid ICP registration, positions of the above landmarks were determined manually on the body surfaces of both the reference model 52 and the patient body model 50. The total objective function E can be solved by the method disclosed in Optimal Step Nonrigid ICP Algorithms for Surface Registration, 2007 IEEE Conference on Computer Vision and Pattern Recognition 1-8, written by Amberg B. Romdhani S, and Vetter T.

### (Simulation)

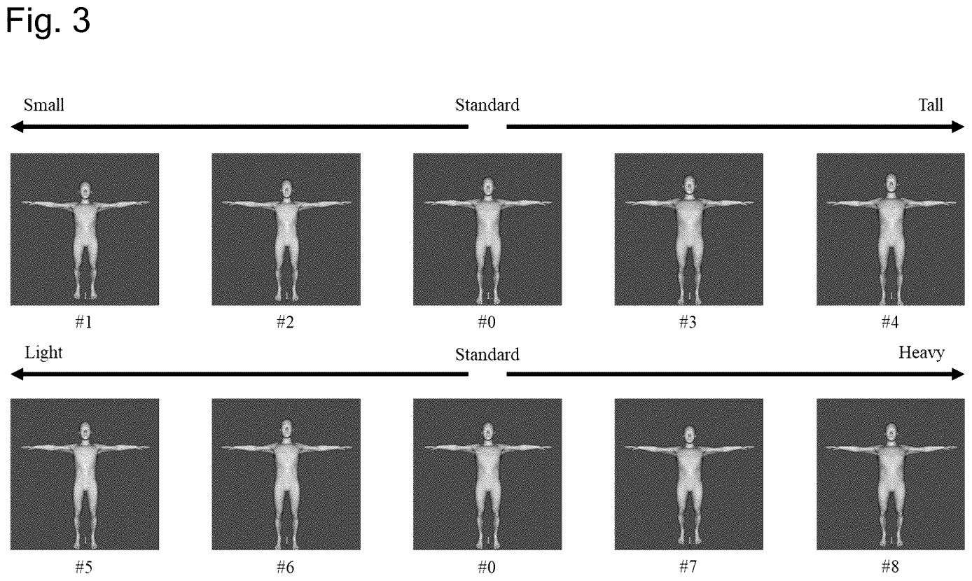

With respect to improvement of accuracy of registration that is to be achieved by selecting a comparison model which closely resembles a patient body 5 (a patient body model 50), the inventors of the medical navigation method and the medical navigation system according to the present embodiment performed verification by performing simulation. For simulation, the inventors constructed some types of human body models by using digital human platform software “DhaibaWorks” (National Institute of Advanced Industrial Science and Technology, https://www.dhaibaworks.com/). The above software makes it possible to generate, as mesh data, human body models having various kinds of shapes. By using the above software, the inventors constructed nine kinds of human body models including shorter models (#1, #2) and taller models (#3, #4), and lighter models (#5, #6) and heavier models (#7, #8), compared with a model (#0) having standard height and weight, as shown in FIG. 3.

In the present simulation, a standard model (#0 in FIG. 3) was fixed as the reference model 52 (the source mesh), and each of other models (#1-#8 in FIG. 3) was set to the patient body model 50 (the target mesh). Thereafter, the non-rigid ICP was applied to each pair comprising the source mesh and each target mesh. Accuracy of the registration was evaluated by using an error between the position of each valve projected by performing registration and the actual position of each valve determined based on medical knowledge of a clinical specialist. Further, the chamfer distance was calculated with respect to each pair comprising the source mesh and each target mesh.

Table 1 is a table which shows result of non-rigid ICP registration errors relating to respective valve positions and chamfer distances in the simulated human body models. The auscultation areas I-IV in Table 1 represent auscultation areas for the aortic valve, the pulmonary valve, the tricuspid valve, and the mitral valve, respectively. According to the result, it is suggested that the registration error becomes large as the deviation between the body shapes of the target model (#1-#8 in FIG. 3) and the reference model (#0 in FIG. 3) becomes large. Further, the chamfer distance represents the degree of the deviation between shapes, and the degree corresponds to the registration error. As a result of the above simulation, it has been proved that accuracy of positioning can be improved by selecting a reference model 52 which closely resembles the patient body 5.

TABLE 1
Non-rigid ICP Registration Error and
Chamfer Distance in Simulation

| | Registration Error in Each Auscultation Area (mm) | | | | | |
|---|---|---|---|---|---|---|
| Human | | | | | Average ± | |
| Body | | | | | Standard | Chamfer |
| Model | I | II | III | IV | Deviation | distance |
| #1 | 40.21 | 29.38 | 44.54 | 33.96 | 37.02 ± 6.70 | 47.15 |
| #2 | 18.99 | 27.40 | 16.44 | 23.58 | 21.60 ± 4.87 | 19.59 |
| #3 | 22.55 | 19.50 | 4.32 | 2.10 | 12.12 ± 10.40 | 3.74 |
| #4 | 13.05 | 14.14 | 26.46 | 16.74 | 17.59 ± 6.11 | 12.98 |
| #5 | 16.90 | 14.55 | 28.61 | 22.36 | 20.61 ± 6.26 | 35.07 |
| #6 | 9.37 | 14.50 | 23.06 | 8.68 | 13.90 ± 6.63 | 9.64 |
| #7 | 23.97 | 15.98 | 5.80 | 18.72 | 16.12 ± 7.67 | 12.34 |
| #8 | 25.49 | 25.71 | 20.17 | 18.11 | 22.37 ± 3.82 | 36.82 |

### (Experiment)

Next, the inventors of the medical navigation method and the medical navigation system according to the present embodiment performed an experiment relating to the medical navigation method according to the present embodiment by using actual human bodies. For the experiment, eight body-surface data sets of eight healthy male volunteers were obtained. The male volunteers were selected by taking a diversity of body shapes into consideration. The average height, weight and body mass index (BMI) of the volunteers were 1.73±0.06 m, 69.1±5.87 kg and 23.2±1.98 kg/m², respectively. Table 1 is a table which shows detailed information of the accepted volunteers.

TABLE 2
Information of Volunteers

| | Height | Weight | Body Mass Index |
|---|---|---|---|
| No. | (m) | (kg) | (kg/m²) |
| #1 | 1.73 | 77 | 25.7 |
| #2 | 1.82 | 78 | 23.5 |
| #3 | 1.73 | 71 | 23.7 |
| #4 | 1.67 | 67 | 24.0 |
| #5 | 1.69 | 69 | 24.2 |
| #6 | 1.65 | 65 | 23.9 |
| #7 | 1.71 | 63 | 21.5 |
| #8 | 1.81 | 63 | 19.2 |

The experiment was performed by using the medical robot device 1 shown in FIG. 1. The point cloud data of the body surface was obtained by using the RGB-D camera 30 (L515 RealSense, Intel, USA). The volunteer laid himself down on a bed, and the RGB-D camera 30 was arranged in a position that is approximately 80 cm above the surface of the bed. Further, similar to the case of the above-explained simulation, the actual auscultation position for each valve on the obtained body surface of each volunteer was determined by a clinical specialist. In the present experiment, the registration errors and the chamfer distances were calculated, and all combinations each registration error and each chamfer distance regarding the body surfaces of the eight volunteers were compared. Body data of a single volunteer was fixed as a source model (a reference model), and pieces of body data of other seven volunteers were set as comparison models (i.e., N=7 herein); and registration between the source model and each comparison model was performed, and the above process was repeated while fixing each of the pieces of the body data of the eight volunteers as the source model.

Table 3 is a table which shows result of non-rigid ICP registration performed between respective pieces of body data of the respective volunteers. The auscultation areas I-IV in Table 3 represent auscultation areas for the aortic valve, the pulmonary valve, the tricuspid valve, and the mitral valve, respectively. In Table 3, the minimum values and the maximum values of the chamfer distances and the average values of registration errors calculated in relation to the respective source models are shown by using characters in bold type (shaded parts). According the result, it can be understood that, in the approximately seven-eighths of source model conditions, the minimum chamfer distance corresponds to the minimum registration error, and the maximum chamfer distance corresponds to the maximum registration error.

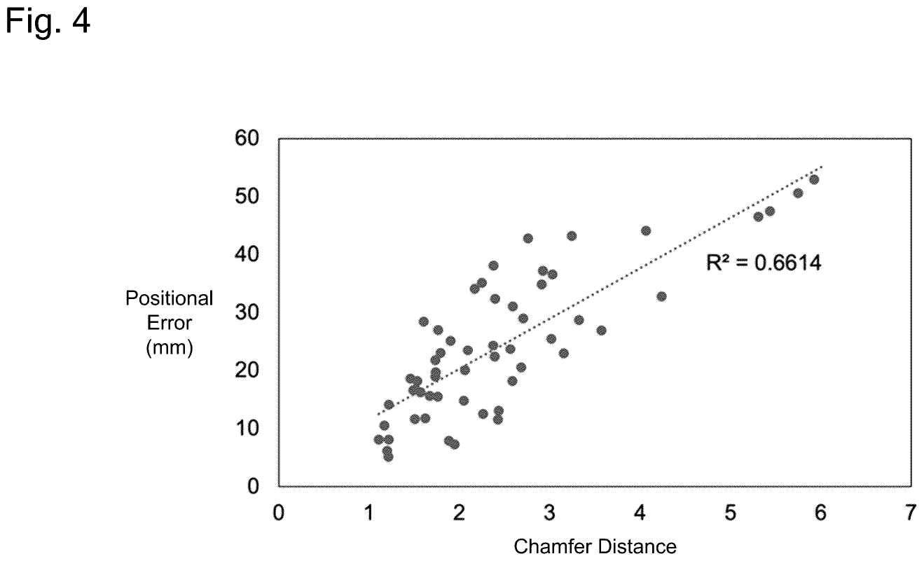

FIG. 4 is a figure which shows all registration errors corresponding to the chamfer distances. In relation to the result shown in FIG. 4, linear regression analyses was performed for checking for the degree of relevancy between accuracy of the non-rigid ICP registration and similarity of the compared models. The liner coefficient of determination R² was approximately 0.66, and this shows that there is some relationship between the chamfer distance and the registration error.

### CONCLUSION

As explained above, the non-rigid ICP registration according to the present example makes it possible to estimate an auscultation area with an average error of 5-19 mm, and provide a more accurate auscultation area that takes individual difference between body shapes into consideration. It is considered that an error in a range less than 20 mm does not affect auscultation qualitatively. Further, through the above-explained simulation and experiment, it has been confirmed that accuracy of registration is dependent on similarity between the body surface of the patient body 5 (the patient body model 50) and the body surface of the reference model 52. The statistical result showed that there is correlation between the accuracy of registration and the chamfer distance equivalent to the similarity of the used models.

Further, in a process in the non-rigid ICP registration, nipples and a navel were used as landmarks. In place of or in addition to the above landmarks, a different part(s) that can be identified easily on a body surface, such as a boundary of a rib or the like, may be used as a landmark(s). By using an additional landmark, accuracy of registration may further be improved.

Further, although the number of comparison models (pieces of data thereof) is seven in the above example, the number is not limited to seven. For example, if it is regarded that Body Shape≈BMI, it is possible to classify body shapes into six types, according to the criterion (that applies to both men and women) that has been defined as shown in the table below by Japan Society for the Study of Obesity and used for judging classes of obesity.

TABLE 4

| BMI value | Judgment |
|---|---|
| Less than 18.5 | Underweight (Skinny) |
| 18.5 to less than 25 | Normal weight |
| 25 to less than 30 | Obese (Class 1) |
| 30 to less than 35 | Obese (Class 2) |
| 35 to less than 40 | Obese (Class 3) |
| 40 or more than 40 | Obese (Class 4) |

Based on the above classification, the number N of comparison models may be set to an integer equal to or greater than 6, or, more preferably, an integer equal to or greater than 12 (six men and six women). In this regard, the above criterion defined by Japan Society for the Study of Obesity is an example, and the number of comparison models may be determined based on a similar medical basis.

### (Process Flow)

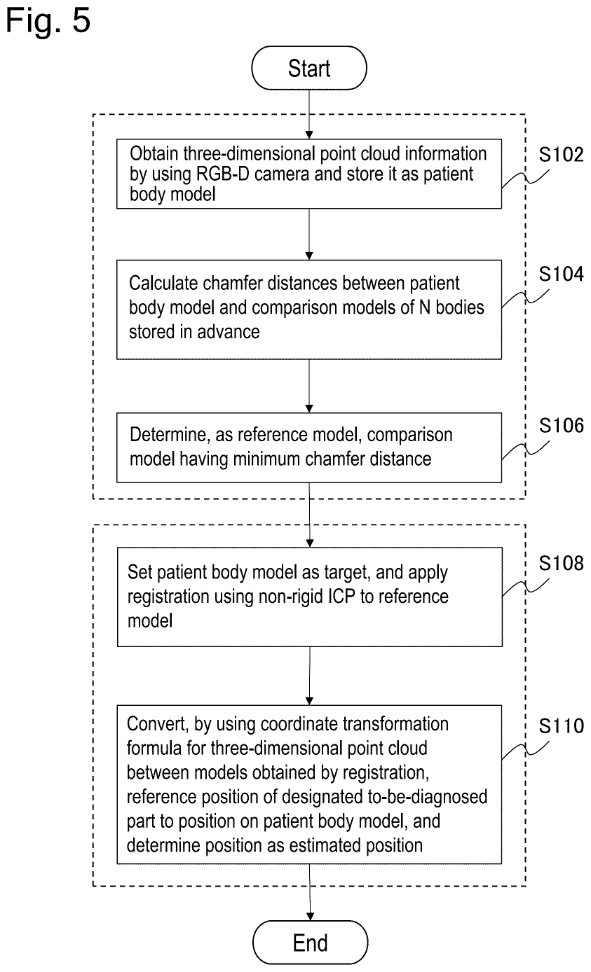

FIG. 5 is a figure which shows an example of a process flow of the medical navigation method according to the present embodiment. The following steps S102-S106 correspond to the processes in step (A) (selecting of a comparison model which most closely resembles a body of a patient) explained in relation to FIG. 2, and steps S108-S110 correspond to the processes in step (B) (non-rigid ICP for body-surface registration) explained in relation to FIG. 2.

In step S102, the RGB-D camera 30 obtains three-dimensional point cloud information of the patient body 5. The following is an example of a method for obtaining the three-dimensional point cloud information of the patient body 5. Specifically, the RGB-D camera 30 moves to a predetermined initial position. The RGB-D camera 30 obtains three-dimensional point cloud information (as referred to “obtained three-dimensional point cloud information”) at respective imaging positions while it moves in an imaging area (the whole breast part of the subject 50 in the case of the present embodiment). Further, at the time, the RGB-D camera 30 obtains two-dimensional color image information in respective imaging parts, and position and angle information of the RGB-D camera 30 in the respective imaging positions (camera position coordinate information). The above processes are repeated until its movement completely covers the whole imaging area. In this regard, the above process is performed by controlling the robot arm 10, the RGB-D camera 30, and so on by using control signals from the client computer device 60. Reconstruction of the shape of the body surface of the subject 50 is performed by using the obtained multiple pieces of three-dimensional point cloud information and multiple pieces of camera position coordinate information that are obtained in multiple imaging positions. More specifically, for example, based on the position and angle information (the position coordinate information) of the RGB-D camera 30 in the respective imaging positions, the obtained multiple pieces of the obtained three-dimensional point cloud information are converted from local coordinates to global coordinates. Correspondence relationship between points that overlap with one another is searched for, in respective pieces of the obtained three-dimensional point cloud information. Based on the correspondence relationship between the overlapping points that has been searched for, the multiple pieces of the obtained three-dimensional point cloud information are unified into a single piece of three-dimensional point cloud information. The three-dimensional point cloud information, that has been obtained as explained above, is stored as data of the patient body model 50 in a storage region such as a hard disk device or the like in the client computer device 60.

In step S104, the client computer device 60 calculates chamfer distances between comparison models 52-k of N bodies, that have been stored in advance in the hard disk device or the like in the client computer device 60, and the body model of the patient 5 obtained in step S102. In step S106, the client computer device 60 determines, as a reference model 52, a comparison model with respect to which the chamfer distance is minimum.

In step S108, the client computer device 60 sets the patient body model 50 as a target, and applies registration using non-rigid ICP to the reference model 52. In step S110, by using coordinate transformation formulas, obtained by registration, for the three-dimensional point cloud between the patient body model 50 and the reference model 52, the client computer device 60 converts reference positions 55, that have been stored in advance in the medical navigation system and are positions of to-be-inspected parts (the aortic valve, the pulmonary valve, the tricuspid valve, and the mitral valve) (positions where a stethoscope should be arranged), to positions on the patient body model 50, and determines them as estimated positions 55 of auscultation positions. For example, the determined estimated position(s) is outputted to be displayed on a remote computer device which is manipulated by a physician and connected to the client computer device 60 via a network. Thereafter, the physician is allowed to manipulate the medical robot device 1 (the robot arm 10) while visually observing the displayed estimated position(s). Alternatively, the estimated position(s) determined in step S110 may be outputted to be displayed on a display of the client computer device 60, and the patient himself/herself, a close relative of the patient, or the like may arrange a stethoscope on the displayed estimated position(s). Further, it is possible to have a construction that the medical robot 1 automatically arranges the stethoscope 40 on the estimated position(s) determined in step S110, obtains auscultation data of the patient 5, and supplies it to a clinical specialist such as a physician or the like, and so on.

In the above description, embodiments of the present invention have been explained, and, in this regard, it is needless to state that the present invention is not limited to any of the above-explained embodiments, and can be implemented in any of various different modes within the scope of the technical idea of the present invention.

For example, although a case wherein a stethoscope is used as a medical device are explained in the above-explained embodiment, the case is not limited to that explained above. It can be used in various cases such as a case of navigation in robot diagnosis and treatment that requires arrangement of the other medical equipment(s) on the patient body 5, a case of preoperative planning, and so on. For example, in ultrasonic examination performed by an autonomous robot by using an ultrasound probe, a scanning path(s) or a scanning area(s) that should be scanned by the ultrasound probe can be estimated on the body surface of the patient 5 while taking individual difference between body shapes, by using the medical navigation method and the medical navigation system according to the present embodiment.

Further, although nipples and a navel are used as landmarks on the body surface of the subject 5 in the above-explained embodiments, it is possible to use, as a landmark(s), a part(S) having noticeable external appearance in a human body. For example, it is considered that it is possible to use a shoulder, a collarbone, a pelvis, or the like as a landmark(s).

The scope of the present invention is not limited by the illustrated embodiments described and shown in the figures, and it comprises all embodiments which provide effect that is equal to that the present invention aims to provide. Further, the scope of the present invention is not limited to that of any of combinations of characteristics of the invention defined by respective claims, and it can be defined by any desired combinations of specific characteristics in all of disclosed respective characteristics.

It should be reminded that the constructions explained below also belong to the scope of the technique of the present invention.

(1) A medical navigation method performed by a computer system for arranging a medical device on a subject, wherein
the computer system holds three-dimensional point cloud information of the subject as body model data of the subject, and holds N pieces of comparison model data showing human body shapes, and arrangement data showing a position(s) for arrangement of the medical device in each of the pieces of comparison model data, and
the medical navigation method comprising:
a step for determining a piece of the comparison model data that has the highest degree of similarity between it and the body model data of the subject, in the N pieces of the comparison model data, as reference model data;
a step for setting, as a target, the body model data of the subject, and applying registration using a non-rigid ICP (interactive closest point) algorithm to the reference model data; and
a step for converting, by using a coordinate transformation formula, obtained as a result of the registration, for the three-dimensional point cloud information between the body model data of the subject and the reference model data, the arrangement data in the reference model data to arrangement data for the medical device in the body model data of the subject.

(2) The medical navigation method in above item (1), wherein the step for determining, as reference model data, a piece of the comparison model data that has the highest degree of similarity between it and the body model data of the subject comprises: calculating a chamfer distance between each of the N pieces of the comparison model data and the body model data of the subject; and determining, as the reference model data, a piece of the comparison model data with respect to that the chamfer distance is minimum.

(3) The medical navigation method in above item (1) or (2), wherein the three-dimensional point cloud information of the subject is obtained by taking images of the subject from multiple imaging positions.

(4) The medical navigation method in any one of above items (1)-(3) further comprising a step for outputting the arrangement data for the medical device, in the body model data of the subject, to a remote computer device.

(5) A medical navigation system for arranging a medical device on a subject, for
holding three-dimensional point cloud information of the subject as body model data of the subject, and holding N pieces of comparison model data showing human body shapes, and arrangement data showing a position(s) for arrangement of the medical device in each of the pieces of the comparison model data;
determining a piece of the comparison model data that has the highest degree of similarity between it and the body model data of the subject, in the N pieces of the comparison model data, as reference model data;
setting, as a target, the body model data of the subject, and applying registration using a non-rigid ICP (interactive closest point) algorithm to the reference model data; and
converting, by using a coordinate transformation formula, obtained as a result of the registration, for the three-dimensional point cloud information between the body model data of the subject and the reference model data, the arrangement data in the reference model data to arrangement data for the medical device in the body model data of the subject.

(6) The medical navigation system in item (5) further comprising an RGB-D camera, wherein the three-dimensional point cloud information is obtained as a result that the RGB-D camera takes images of the subject from multiple imaging positions.

(7) A computer program which makes a computer system perform the medical navigation method in any one of above items (1)-(4),

(8) A computer-readable storage medium in which a computer program which makes a computer system perform the medical navigation method in any one of above items (1)-(4) is stored.

### REFERENCE SIGNS LIST

1 . . . Medical robot device (Medical navigation system)
5 . . . Patient (Inspection object)
10 . . . Robot arm
20 . . . Constant force passive scan mechanism (End effector)
25 . . . Force and torque sensor
30 . . . LIDAR camera
40 . . . Medical device (Stethoscope)
50 . . . Patient body model
55 . . . Reference position
56 . . . Estimated position
60 . . . Client computer device

1. A medical navigation method performed by a computer system for arranging a medical device on a subject, wherein
the computer system holds three-dimensional point cloud information of the subject as body model data of the subject, and holds N pieces of comparison model data showing human body shapes (N is an integer equal to or greater than 2), and arrangement data showing a position for arrangement of the medical device in each of the pieces of the comparison model data, and
the medical navigation method comprising:
a step for determining a piece of the comparison model data that has the highest degree of similarity between it and the body model data of the subject, in the N pieces of the comparison model data, as reference model data;
a step for setting, as a target, the body model data of the subject, and applying registration using a non-rigid ICP (interactive closest point) algorithm to the reference model data; and
a step for converting, by using a coordinate transformation formula, obtained as a result of the registration, for the three-dimensional point cloud information between the body model data of the subject and the reference model data, the arrangement data in the reference model data to arrangement data for the medical device in the body model data of the subject,
wherein the step for determining, as reference model data, a piece of the comparison model data that has the highest degree of similarity between it and the body model data of the subject includes: calculating a chamfer distance between each of the N pieces of the comparison model data and the body model data of the subject, the each of the N pieces of the comparison model data being applied in advance with registration using an ICP algorithm, in which the body model data of the subject is set as a target; and determining, as the reference model data, a piece of the comparison model data with respect to that the chamfer distance is minimum.

2. (canceled)

3. The medical navigation method as recited in claim 1, wherein the three-dimensional point cloud information of the subject is obtained by taking images of the subject from multiple imaging positions.

4. The medical navigation method as recited in claim 1 further comprising a step for outputting the arrangement data for the medical device, in the body model data of the subject, to a remote computer device.

5. The medical navigation method as recited in claim 1, wherein the medical device is a stethoscope or an ultrasound probe.

6. A medical navigation system for arranging a medical device on a subject, for
holding three-dimensional point cloud information of the subject as body model data of the subject, and holding N pieces of comparison model data showing human body shapes, and arrangement data showing a position for arrangement of the medical device in each of the pieces of the comparison model data;
determining a piece of the comparison model data that has the highest degree of similarity between it and the body model data of the subject, in the N pieces of the comparison model data, as reference model data;
setting, as a target, the body model data of the subject, and applying registration using a non-rigid ICP (interactive closest point) algorithm to the reference model data; and
converting, by using a coordinate transformation formula, obtained as a result of the registration, for the three-dimensional point cloud information between the body model data of the subject and the reference model data, the arrangement data in the reference model data to arrangement data for the medical device in the body model data of the subject,
wherein the determining, as reference model data, a piece of the comparison model data that has the highest degree of similarity between it and the body model data of the subject includes: calculating a chamfer distance between each of the N pieces of the comparison model data and the body model data of the subject, the each of the N pieces of the comparison model data being applied in advance with registration using an ICP algorithm, in which the body model data of the subject is set as a target; and determining, as the reference model data, a piece of the comparison model data with respect to that the chamfer distance is minimum.

7. The medical navigation system as recited in claim 6 further comprising a robot arm and an RGB-D camera that is attached to the point of the robot arm, wherein
multiple obtained three-dimensional point cloud information, that are multiple pieces of the three-dimensional point cloud information of the subject, is obtained as a result that the RGB-D camera takes images of the subject from multiple imaging positions as the robot arm moves, and the multiple three-dimensional point cloud information of the subject is obtained based on correspondence relationship between points overlapping with one another in the multiple pieces of the obtained three-dimensional point cloud information.

8. A computer-readable storage medium in which a computer program which makes a computer system perform the medical navigation method recited in claim 1 is stored.
